(19) United States
(12) Patent Application Publication
(10) Pub. No.: US 2024/0415658 A1
(43) Pub. Date: Dec. 19, 2024

(54) IMPLANTABLE MEDICAL DEVICE WITH TETHERED POWER AND COMMUNICATION INTERFACE

(71) Applicant: Boston Scientific Scimed, Inc., Maple Grove, MN (US)

(72) Inventors: Brian P. Watschke, Minneapolis, MN (US); Jean M. Bobgan, Maple Grove, MN (US)

(21) Appl. No.: 18/733,375

(22) Filed: Jun. 4, 2024

Related U.S. Application Data
(60) Provisional application No. 63/508,774, filed on Jun. 16, 2023.

(51) Int. Cl. A61F 2/26; A61F 2/00

(52) U.S. Cl. CPC A61F 2/26; A61F 2/004

(57) ABSTRACT

An implantable medical device is disclosed. The implantable medical device includes a housing forming an internal compartment. Electronic components are disposed within the internal compartment. An interface system receives power signals, wireless electrical communication signals, or both. An elongated tether having a conductive lead mechanically couples the housing to the interface system and allows the housing and interface system to be implanted at spaced-apart locations within the patient. The conductive lead electrically couples the interface system to the electronic components.

### CROSS-REFERENCE TO RELATED APPLICATION

This application claims priority to U.S. Provisional Patent Application No. 63/508,774, filed on Jun. 16, 2023, entitled “IMPLANTABLE MEDICAL DEVICE WITH TETHERED POWER AND COMMUNICATION INTERFACE”, the disclosure of which is incorporated by reference herein in its entirety.

### TECHNICAL FIELD

The present disclosure relates generally to medical systems and implantable medical devices. More specifically, the present disclosure relates to implantable medical devices that recharge and communicate in medical systems.

### BACKGROUND

Implantable medical devices include electrical or electromechanical medical devices that are implanted within a patient and perform a task such as to monitor a parameter of the patient or to deliver a therapy to the patient via electrical energy. For example, the implantable medical device can be an implantable urological device implanted into the patient to treat a condition such as erectile disfunction, penile deformity, or incontinence. Some implantable medical devices are designed to receive communication signals from external devices of a medical system. Many implantable medical devices are designed to receive power directly from an energy storage system such as a battery or capacitor located with the implantable medical device, but the energy storage system can be depleted of energy long before the end of the useful life of the implantable medical device. In some examples, the implantable medical device includes a rechargeable energy storage system such as a rechargeable battery to extend the life of the implantable medical device. A wireless charger may be applied to recharge a depleted battery in the implanted medical device.

### SUMMARY

In Example 1, an implantable medical device comprising a housing forming an internal compartment, electronic components disposed within the internal compartment, an interface system configured to receive at least one of power signals wireless electrical communication signals, and an elongated tether having a conductive lead, wherein the tether mechanically couples the housing to the interface system and allows the housing and interface system to be implanted at spaced-apart locations within a patient, further wherein the conductive lead electrically couples the at interface system to the electronic components.

In Example 2, the implantable medical device of Example 1, wherein the implantable medical device is included in an implantable urological device.

In Example 3, the implantable medical device of Example 2, wherein the implantable urological device is an inflatable penile prosthesis.

In Example 4, the implantable medical device of any of Examples 2-3, wherein the implantable urological device includes a reservoir having a fluid, and the implantable medical device is in fluid communication with the reservoir.

In Example 5, the implantable medical device of Example 4, wherein the implantable urological device includes a plurality of inflatable cylinders to receive the fluid, and the implantable medical device is in fluid communication with the plurality of inflatable cylinders, and wherein the implantable medical device is configured to pump the fluid from the reservoir to the plurality of inflatable cylinders.

In Example 6, the implantable medical device of any of Examples 1-5, wherein the implantable medical device is included in a medical system further comprising a remote charger and a remote programmer.

In Example 7, the implantable medical device of Example 6, wherein the remote charger is operably coupleable to the interface system to provide the transcutaneous inductive power transfer.

In Example 8, the implantable medical device of any of Examples 6-7, wherein the programmer is a handheld programmer in radiofrequency communication with the electronic components via the interface system.

In Example 9, the implantable medical device of Example 8, wherein the programmer includes a software application running on a mobile computing device to operate the implantable medical device.

In Example 10, the implantable medical device of any of Examples 1-9, and further comprising a rechargeable power source coupled to the electronic components and disposed within the internal compartment.

In Example 11, the implantable medical device of Example 10, wherein the rechargeable power source is a battery disposed within the internal compartment.

In Example 12, the implantable medical device of any of Examples 1-11, and further comprising a fluidic circuit disposed within the internal compartment.

In Example 13, the implantable medical device of any of Examples 1-12, wherein the interface system includes a coil configured to receive the power signals and an antenna configured to receive the wireless electrical communication signals.

In Example 14, the implantable medical device of Example 13, wherein the interface system includes the coil and the antenna disposed within a ceramic puck.

In Example 15, the implantable medical device of Example 14, and further comprising a mesh coupled to the puck.

In Example 16, an implantable medical device comprising a housing forming an internal compartment, electronic components disposed within the internal compartment, an interface system configured to receive at least one of power signals wireless electrical communication signals, and an elongated tether having a conductive lead, wherein the tether mechanically couples the housing to the interface system and allows the housing and interface system to be implanted at spaced-apart locations within a patient, further wherein the conductive lead electrically couples the at interface system to the electronic components.

In Example 17, the implantable medical device of Example 16, wherein the electronic components include a treatment system, a communication system, and a recharge system, and the recharge system is coupled to a rechargeable power source.

In Example 18, the implantable medical device of Example 17, wherein the rechargeable power source is a battery disposed within the internal compartment.

In Example 19, the implantable medical device of Example 16, and further comprising a fluidic circuit disposed within the internal compartment.

In Example 20, the implantable medical device of Example 19, wherein the fluidic circuit includes a pump assembly in fluid communication with a valve assembly and a pressure sensor.

In Example 21, the implantable medical device of Example 19, wherein the internal compartment includes a plurality of partitions including a first partition and a second partition, the fluidics circuit disposed within the first partition and the electronics components disposed in the second partition, wherein the first partition is hermetically sealed from the second partition.

In Example 22, the implantable medical device of Example 21, wherein the tether includes a proximal end and a distal end, wherein the distal end is mechanically coupled to the interface system and the proximal end is received within a partition of the plurality of partitions and mechanically coupled to the housing.

In Example 23, the implantable medical device of claim of Example 22, wherein the conductive lead is electrically coupled to electronic components via feedthrough pins coupled to the proximal end.

In Example, 24, the implantable medical device of Example 16, wherein the interface system includes a coil configured to receive the power signals and an antenna configured to receive the wireless electrical communication signals.

In Example 25, the implantable medical device of Example 24, wherein the interface system includes the secondary conductor and the antenna disposed within a ceramic puck.

In Example 26, the implantable medical device of Example 25, wherein the puck includes a first major surface and an opposite second major surface, and wherein the first major surface is coupled to a mesh layer.

In Example 27, the implantable medical device of Example 26, wherein the second major surface is textured to form a protrusion.

In Example 28, the implantable medical device of Example 25, wherein coil and antenna are disposed within a flexible puck formed of a polyurethane or silicone.

In Example 29, the implantable medical device of Example 16, wherein the housing is configured to be implanted in a first location of a patient and the interface system is configured to be implanted in a second location of the patient.

In Example 30, a method to implant an implantable medical device within a patient. The method comprising providing an implantable medical device, the implantable medical device including a housing forming an internal compartment, electronic components and a rechargeable power source disposed within the internal compartment, the electronic components including a treatment system, a communication system and a recharge system, the recharge system coupled to the rechargeable power source, an interface system including at least one of a coil configured to receive power signals and an antenna configured to receive wireless electrical communication signals, and an elongated tether having a conductive lead, wherein the tether mechanically couples the housing to the interface system and allows the housing and interface system to be implanted at spaced-apart locations within a patient, further wherein the conductive lead electrically couples the at least one of the coil and the antenna to the electronic components, implanting the housing in a first location within the patient, and implanting the interface system at a second location with the patient and spaced-apart from the first location.

In Example 31, the method of Example 30, wherein the tether is integrally formed with the interface device, and further comprising coupling the tether to the electronic components.

In Example 32, implantable urological device, comprising a reservoir configured to receive a fluid, an inflatable member in fluid communication with the reservoir, an implantable medical instrument in fluid communication with the reservoir and the inflatable member, the implantable medical instrument comprising a housing forming an internal compartment, a fluidic circuit disposed within the internal compartment, the fluidic circuit including a pump assembly configured to transfer the fluid from the reservoir to the inflatable member, and electronic components operably coupled to the fluidic circuit and configured to operate the fluidic circuit, the electronic components coupled to a rechargeable power source, the electronics components disposed within the internal compartment, wherein the electronic components include a communication system and a recharge system disposed within the internal compartment, the recharge system coupled to the rechargeable power source, an interface system including at least one of a coil and an antenna, the coil configured to receive power signals, and the antenna configured to receive electrical communication signals, and an elongated tether having a conductive lead, wherein the tether mechanically couples the housing to the interface system and allows the housing and interface system to be implanted at spaced-apart locations within the patient, further wherein the conductive lead electrically couples the at least one of the coil and the antenna to the electronic components.

In Example 33, the implantable urological device of Example 32, wherein the inflatable member includes a plurality of inflatable cylinders in fluid communication with the medical instrument and the reservoir, the plurality of inflatable cylinders configured to be disposed within a corpora cavernosa of a penis.

In Example 34, the implantable urological device of Example 32, wherein the medical instrument is configured to be disposed within a retropubic space. In other examples, the medical instrument may be placed between the transverse muscle and the rectus muscle.

In Example 35, the implantable urological device of Example 32 wherein the interface system is integrally formed with the tether, and the tether is removably coupleable to the medical instrument.

In another example of an implantable urological device, the inflatable member includes an inflatable cuff, which may be implemented as an artificial urinary sphincter.

While multiple embodiments are disclosed, still other embodiments of the present disclosure will become apparent to those skilled in the art from the following detailed description, which shows and describes illustrative embodiments of the disclosure. Accordingly, the drawings and detailed description are to be regarded as illustrative in nature and not restrictive.

### BRIEF DESCRIPTION OF THE DRAWINGS

FIG. 1 is a schematic diagram illustrating an example implantable medical system including an example implantable medical device, an example external charger, and an example programmer of the present disclosure.

FIG. 2 is a perspective view of an example implantable urologic device including features of the example implantable medical device of FIG. 1.

FIG. 3 is a block diagram illustrating example components of the example implantable medical device of FIG. 2.

FIG. 4 is a perspective view of a feature of the example implantable medical device of FIG. 2.

FIG. 5 is a perspective view of another feature of the example implantable medical device of FIG. 2.

FIG. 6 is a perspective view of an example inflatable member.

While the disclosure is amenable to various modifications and alternative forms, specific embodiments have been shown by way of example in the drawings and are described in detail below. The intention, however, is not to limit the disclosure to the particular embodiments described. Rather, the disclosure is intended to cover all modifications, equivalents, and alternatives falling within the scope of the disclosure as defined by the appended claims.

### DETAILED DESCRIPTION

For purposes of promoting an understanding of the principles of the present disclosure, reference is now made to the examples illustrated in the drawings, which are described below. The illustrated examples disclosed herein are not intended to be exhaustive or to limit the disclosure to the precise form disclosed in the following detailed description. Rather, these exemplary embodiments were chosen and described so that others skilled in the art may use their teachings. It is not beyond the scope of this disclosure to have a number (e.g., all) of the features in an example used across all examples. Thus, no one figure should be interpreted as having any dependency or requirement related to any single component or combination of components illustrated therein. Additionally, various components depicted in a figure may be, in examples, integrated with various ones of the other components depicted therein (or components not illustrated), all of which are within the ambit of the present disclosure.

Wireless power transfer or transmission is used to deliver power from a power source without a mechanical connection to electronic devices. Wireless power transfer systems are used in a variety of applications, such as, for recharging batteries in mobile computing devices such as smart phones or wearable devices. Wireless power transfer systems are also used to transmit power transcutaneously, or through the skin, to medical devices implanted in a patient either to directly power the implanted medical device or to recharge an energy storage system of the implanted medical device. Examples of wireless power transfer systems include inductive charging and radiofrequency (RF) charging.

In some examples, transcutaneous charging is performed via inductive power transfer or transmission, which is presented here for illustration. The energy storage system of the implantable medical device can be recharged with an external charger configured to provide inductive power transfer. Inductive power transfer can be performed with an inductive coupling between conductors, which may be formed as coils of wire, such as a primary conductor, which can include a primary coil, in the charger and a secondary conductor, such as for example a secondary coil, in the implantable medical device. Power is transferred between the conductors with a magnetic field. An alternating current through the primary conductor creates an oscillating magnetic field. The magnetic field passes through the secondary conductor, and the magnetic field induces an alternating electromotive force, or EMF, such as voltage, which creates an alternating current in the secondary conductor. The induced alternating current may either directly drive a load in the implantable medical device or be rectified to direct current by a rectifier in the implantable medical device, which drives the load. Resonant inductive coupling is a type of inductive coupling in which power is transferred by magnetic fields between two resonant circuits, one in the charger and one in the implantable medical device. Each resonant circuit can include a coil of wire connected to a capacitor, or a self-resonant coil or other resonator with internal capacitance. Resonant circuits, or tank circuits, are tuned to resonate at generally the same resonant frequency. The resonance between the conductors can greatly increase coupling and power transfer between the charger and the implantable medical device. In this example, the external charger does not mechanically connect with the implantable medical device, and the external charger can be used to charge the implantable medical device from a relatively short distance away.

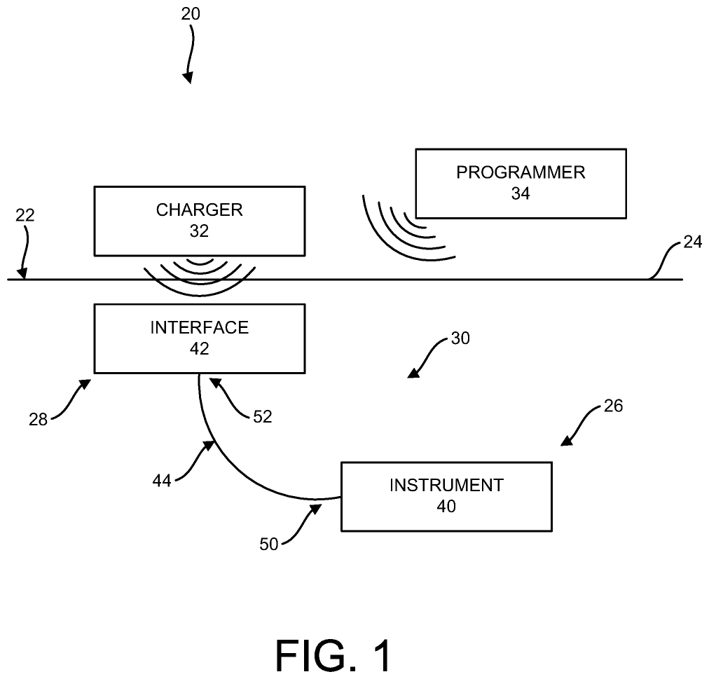

FIG. 1 illustrates an implantable medical device system 20. The implantable medical system 20 includes an implantable medical device 30, which can be fully implanted within a patient 22. The implantable medical device 30 can include a housing forming an internal compartment. The implantable medical device 30 can include an energy storage system, such as a rechargeable power source, and electronic components within the internal compartment of the implantable medical device 30. Examples of the rechargeable power source can include a rechargeable battery, an energy storage component such as a capacitor, or circuitry to apply power received from a transcutaneous power source. The implantable medical device 30 includes a treatment system, which is configured to apply energy from the battery such as perform a task such as to monitor a parameter of the patient or to deliver a therapy to the patient. In the example, the electronics components can include a recharge system and a communication system.

The implantable medical system 20 also includes a charger 32, which can also be referred to as a wireless recharger, outside of the patient 22, or across a transcutaneous boundary 24 such as the surface of the patient's skin proximate the implantable medical device 30. In one example, the charger 32 is placed against the patient 22 and proximate the implantable medical device 30 to inductively transfer energy and to replenish the battery of the implantable medical device 30. The charger 32 can include a primary conductor, such as a coil, to inductively couple with a secondary conductor in the implantable medical device 30 and provide an inductive power transfer to recharge the battery via the recharge system when placed proximate the implantable medical device 30.

The charger 32 is available in different configurations depending on recharge frequencies and communication schemes for use with the implantable medical device 30. For example, a first configuration of the charger 32 may support a bidirectional inductive telemetry communication scheme and a first recharge frequency, a second configuration of the charger 32 may support a radiofrequency telemetry and downlink inductive telemetry communication schemes and a second recharge frequency, and a third configuration of the charger may support the bidirectional inductive telemetry communication scheme and a third recharge frequency.

In certain embodiments, the charger 32 delivers magnetic energy to a corresponding implantable device 30 at the preselected frequency with a resonant inductor-capacitor (LC) tank circuit to generate an H-field. In various embodiments, the tank circuit includes a recharge coil in series with a recharge capacitor. Various configurations of the charger 32 can share a common coil design, and the preselected recharge frequency is determined via a selected tank capacitance of the recharge capacitor. The tank circuit can oscillate at a resonant frequency. A phase locked loop in the tank circuit is created via pulsing an applied tank voltage in phase with a tank current. During resonance, the tank current is approximately or generally sinusoidal over time. The tank circuit can achieve maximum tank power when a tank voltage pulse is aligned in time with the tank current. Recharge power can be adjusted by altering the magnitude and duty of the tank voltage pulse input to the tank circuit.

System 20 can also include a handset programmer 34 configured to wirelessly interface with the implantable medical device 30 or with the charger 32. In one example, the handset programmer 34 can be implemented as a general-purpose computing device or mobile computing device that hosts a software application. For instance, the handset programmer can include a set of controls to transcutaneously communicate with or operate the implantable medical device 30 via the communication system or to communicate with or operate the charger 32.

Systems of the present disclosure can optionally include additional components. System 20 can include a charging dock, which can be plugged into a wall outlet and configured to charge an internal battery of the charger 32. The charger 32 can also be used in conjunction with a fixation product of system 20 to keep the charger 32 in position proximate the implantable medical device 30 during a recharge session. The fixation product can include a fixation belt to be worn around a portion of the patient 22 such as the belt line for an implantable medical device 30 in the abdomen, back, buttocks, or flank of the patient 22, or a fixation drape to be worn around the neck with a counterweight to balance the charger 32 for an implantable medical device 30 in the pectoral region of the patient 22. The fixation product receives the charger 32 to hold the charger 32 in place with respect to the fixation product so that the charger 32, in one example, does not rotate and generally does not move with respect to the implantable medical device 30 during the recharge session and to secure the charger 32 so as not to fall out unless purposefully removed from the fixation product.

In certain applications, an implantable medical device may be deeply embedded in a patient or imbedded in a patient that may not be conducive to inductive charging and wireless communication or in an area of the body where it is relatively uncomfortable to wear the charger during charging. In such circumstances, the clinician may be faced with a dilemma as to how to implant a medical device. For example, the clinician can implant a medical device into the patient to receive effective therapy and compromise on communication or charging capabilities or comfort, or the clinician can implant a medical device to receive effective communication and charging capabilities and comfort but compromise on an effective location to deliver therapy. With the implantable medical device 30 of system 20, however, a clinician can implant the device 30 in locations without having to compromise therapy, charging, or communication capabilities and comfort.

The implantable medical device 30 includes an implantable instrument 40, an implantable remote interface system 42, and an implantable elongated tether 44 that electrically and mechanically connects to the remote interface system 42 to the instrument 40. The implantable instrument 40 includes a housing forming an internal compartment. An energy storage system such as a rechargeable battery, electronic components such as a communication circuit or system, a recharge circuit or system, and circuitry or system to effect therapy or process parameters, and electrical or electromechanical devices to deliver a therapy or detect a parameter are included within the internal compartment of the implantable instrument 40. The interface system 42 includes at least one of a coil configured to receive power signals and an antenna configured to receive wireless electrical communication signals. In one example, the interface system 42 includes coil configured to receive power signals. In another example, the interface system includes both the coil configured to receive power signals and the antenna to receive wireless electrical communication signals. The elongated tether 44 includes a conductive lead, wherein the tether 44 mechanically couples the housing to the interface system 42 and the conductive lead electrically couples the at least one of the coil and the antenna to the electronic components.

Upon implantation in the patient, the remote interface system 42 is spaced apart from the instrument 40. The remote interface includes one or more antennae or secondary conductors to wirelessly interface with the charger 32, the handset programmer 34, or both. For example, the remote interface system 42 is configured to transcutaneously receive power (e.g., inductive or RF) transferred from the charger or to transcutaneously receive or transmit wireless communication via electrical signals with the charger 32 or the handset programmer 34.

The elongated tether 44 includes a first end 50 mechanically coupled to the instrument 40 and a second end 52 mechanically coupled to the remote interface system 42. The tether 44 also carries a lead, such as a plurality of leads, along the body portion 54 to electrically couple the one or more antennae or secondary conductors in the interface system 42 to the electronic components of the instrument to transmit communication and power signals from the interface system 42 to the instrument 40, such as communication and power signals from the charger 32 or communication signals from the handset programmer 34. In some examples, the lead can also transmit communication signals from the instrument 40 to the interface system 42, such as communication signals directed to the handset programmer 34 or the charger 32.

The implantable medical device 30 can be implanted subcutaneously with a plurality of implantation locations or pockets within a patient 22, such as a first location 26 for the instrument 40 and a second location 28, remote and spaced-apart from the first location 26, for the interface system 42. In some examples, another component of the implantable medical device 30 can occupy an additional location within the patient 22. The implantable medical device 30 may be configured to deliver therapy to a patient, monitor parameters within a patient, receive and deliver signals with a patient, such as at regular intervals, continuously, or in response to a detected event such as an event detected by sensors, received from another implantable device (not shown), or received from components of the implantable medical system 20 such as from charger 32 or from handset programmer 34. The implantable medical device 30 can be configured to detect a variety of physiological signals that may be used in connection with various diagnostics, therapeutic, and other monitoring implementations. The implantable medical device 30 can be used in urological, neurological, cardiac, and other applicable fields that apply implantable medical devices with power systems or for receiving and transmitting signals.

In certain embodiments, the implantable medical device 32 is configured as a urological therapy device to deliver selective stimulation to, for example, the sacral nerves for treatment of urological disorders such as bladder control or erectile disorders. In other examples, the implantable medical device is configured as a gastrointestinal device to treat, for instance, gastroesophageal reflux disease (GERD). In other examples, the implantable medical device 32 may be configured as a neurostimulation therapy device for pain management and the like. In still other examples, the implantable medical device 32 may be a cardiac rhythm management device for sensing and stimulating cardiac tissue for treatment of cardiac arrhythmias such as bradycardia, tachycardia and for cardiac resynchronization therapy. In still other examples, the implantable medical device 32 may be configured as a monitoring device only, with no therapeutic functionality, to monitor physiological parameters of a patient. In short, the present disclosure is not limited to any clinical application, and any implantable device that requires power to operate as intended.

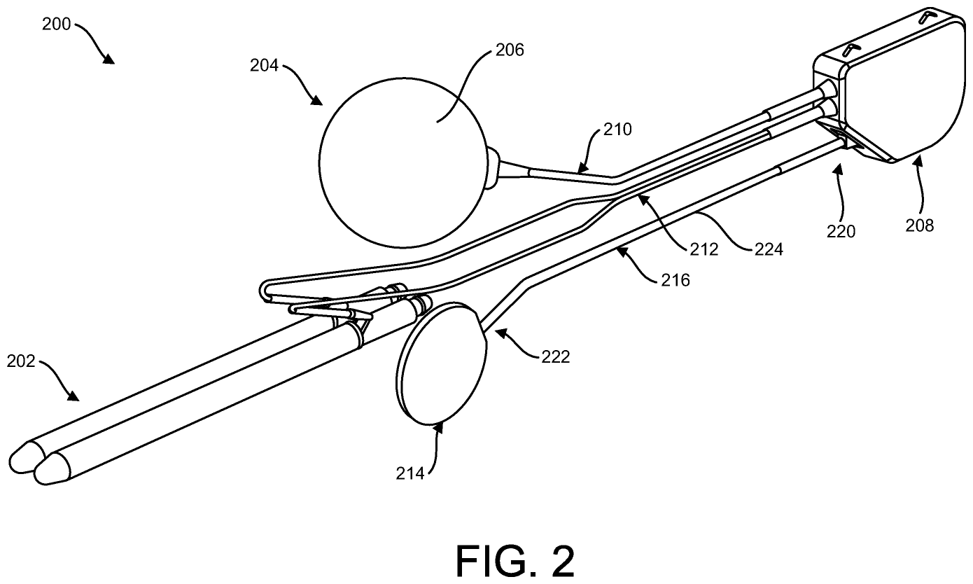

FIG. 2 illustrates an example implantable medical device 200, which can correspond with the implantable medical device 32. In one example, the implantable medical device is an implantable urologic device in connection with an inflatable member, such as a penile prosthesis or inflatable artificial sphincter. The example implantable medical device 200 includes an inflatable member, such as a pair of inflatable cylinders 202, a reservoir 204 that may be filled with a fluid, such as a sterile saline solution 206, and an electromechanical actuation device 208 in a closed system. The reservoir 204 is fluidically coupled to the actuation device 208 via tubing 210, and the actuation device 208 is fluidically coupled to the cylinders 202 via tubing 212. For example, tubing 210, 212 can be kink-resistant tubing made from a silicone elastomer. The actuation device 208 can include an internal fluidic circuit to fluidically couple the reservoir 204 to the cylinders 202 via tubing 210, 212. A power and communication interface system 214 is mechanically coupled to the electromechanical actuation device 208 via tether 216. In one example, the electromechanical actuation device 208 can correspond with instrument 40, the power and communication interface system 214 can correspond with interface system 42, and tether 216 can correspond with tether 44 of FIG. 1.

The cylinders 202 are typically implanted in the corpora cavernosa of the penis, and the reservoir 204 are often implanted in the retropubic space, or Retzius space, of the patient or between the transverse muscle and the rectus muscle. The electromechanical actuation device 208 can be implanted in the abdomen at a selected location determined by a clinician. The sterile saline solution 206 can be pumped from the reservoir 204 into the chambers of the cylinders 202 via tubing 210, 212 with the actuation device 208. For example, the actuation device 208 may include electromechanical pumps and valves within the fluid circuit. The cylinders 202 replace non-functioning or limited-functioning erectile tissue in the penis, and the cylinders 202 inflated with the saline solution 206 produce an erection. After sexual intercourse, the saline solution 206 in the cylinders 202 is returned to the reservoir 204 via the tubing and fluidic circuit within the actuation device 208.

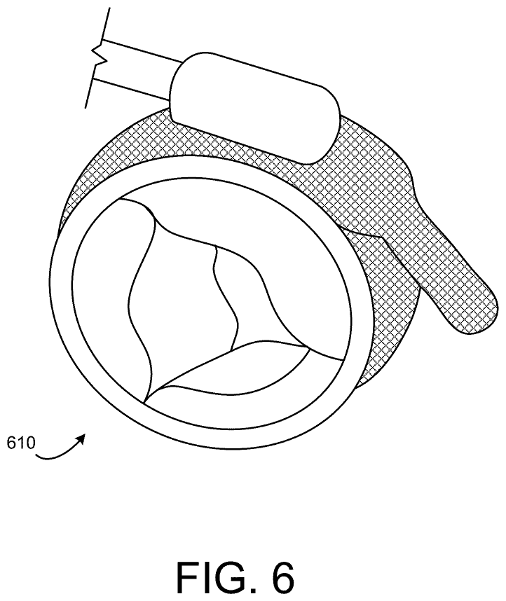

In another example of an implantable medical device incorporating the features of an electromechanical actuation device incorporating features of medical device 30, the inflatable member includes an inflatable cuff, which may be implemented as an artificial urinary sphincter. FIG. 6 illustrates an example inflatable cuff 610. The inflatable cuff 610 is or may be disposed about a urethra proximate the bladder. The actuation device is applied to pump fluid from a reservoir to expand the cuff 610 and to close the urethra. The cuff 610 is deflated to allow a patient to void the bladder. In still another example of an implantable medical device incorporating the features of an electromechanical actuation device incorporating features of medical device 30, the inflatable member includes an inflatable cuff, which may be implemented in an esophagus or other location in the body, such as to treat GERD.

The elongated tether 216 is formed of a biocompatible material and includes a proximal end 220 mechanically coupled to the actuation device 208, a distal end 222 mechanically coupled to the interface system 214, and a body 224. In some embodiments, the proximal end 220 includes electrical contacts or other conductors to electrically couple the tether 216 to the electrical components within the actuation device 208. In various embodiments, the distal end 220 includes electrical contacts or other conductors to electrically couple the tether 216 to electrical conductors such as a secondary conductor or coil and antenna in the interface system 214. In one example, each conductor on the proximal end 220 is coupled to a conductor on the distal end 222 via an elongated lead conductor extending the length of the body 224. For example, one lead conductor may be configured to carry power signals and another lead conductor may be configured to carry communication signals. The plurality of lead conductors can be insulated from one another within an insulating sheath along the body 224, such as with an insulating polymer sheath.

In one example, the actuation device 208 includes a rechargeable battery system to operate the fluid circuit, which can be operated via control signals. The power and communication interface system 214 can be placed at a selected subcutaneous location on the patient to receive inductive power signals from a charger and communication signals from a handset programmer, which can then be transferred to the actuation device 208 via tether 216. For instance, the actuator device 208 can be configured to receive power signals from the interface system 214 to recharge the battery and can be configured to receive communication signals from the interface system 214 to operate the fluidic circuit such as to pump saline solution 206 from the reservoir 204 into the cylinders 202. In one example, a charger, such as charger 32 can provide power signals and communication signals to the interface system 214, and a handheld programmer, such as handheld programmer 34, can provide communication signals to the interface system 214.

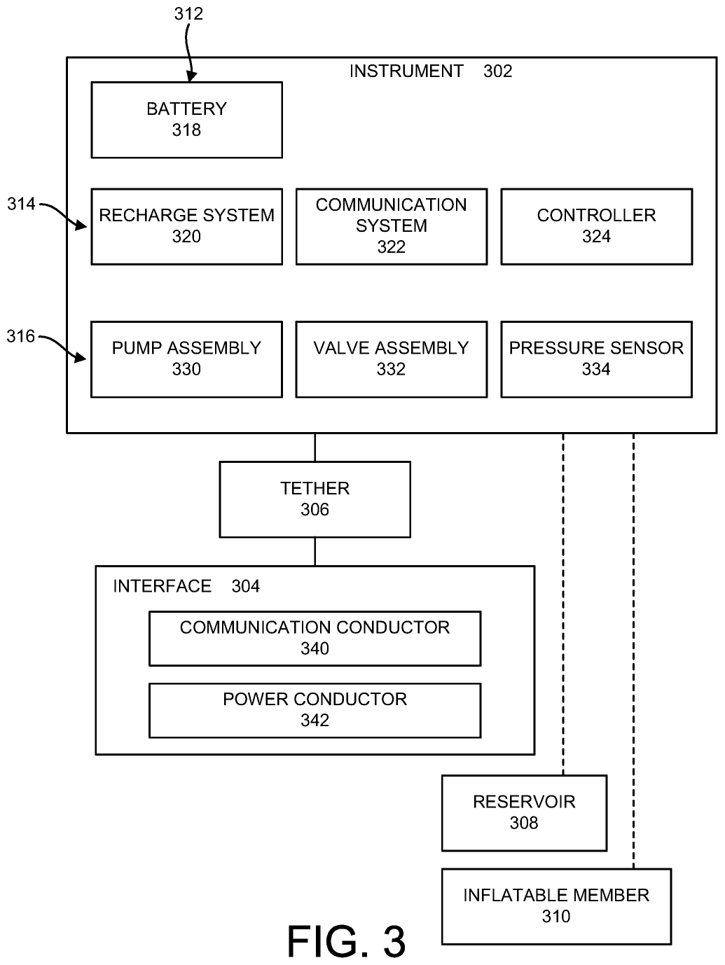

FIG. 3 illustrates components of a medical device 300, which may correspond with the medical device 30. Medical device 300 includes an implantable medical instrument 302, an implantable remote interface system 304, and an implantable elongated tether 306 that electrically and mechanically connects to the remote interface 304 to the instrument 302. In one example, the medical device is a urological therapy device, and includes a reservoir 308 and an inflatable member 310, which are fluidically coupled together via the medical instrument 302.

The implantable instrument 302 includes a housing forming an internal compartment. The internal compartment includes a rechargeable energy storage system 312, electronics components 314, and a fluidic circuit 316. The energy storage system 312 can include a rechargeable battery 318. The rechargeable battery can assume various forms appropriate to provide power for generating desired electrical signals and to store power provided from the recharge circuitry. For example, the battery can incorporate lithium-ion (Li+) chemistry, i.e., a lithium-ion battery.

The electronics components 314 can be operated to effect therapy or process parameters and cause the electrical or electromechanical devices to deliver a therapy or detect a parameter from within the internal compartment as a treatment system. In one example, the electronics can be disposed on a printed circuit board. The electronics components 314 can include a recharge system 320, a communication system 322, and a controller 324. The recharge system 320 includes hardware configured to interface with the tether 306 within the internal compartment to receive power signals, and to provide the power signals in a form suitable to recharge the energy storage system 312 and can include circuitry to reduce the likelihood of overcharging. The communication system 322 includes hardware configured to interface with the tether 306 within the internal compartment to receive electrical communication signals. For instance, the implantable device 300 can be configured to communicate via a wireless personal area network technology such as Bluetooth Low Energy, which is compatible with several operating systems that can be applied in mobile devices configured as handset programmers. Communication system 322 can include an integrated circuit to implement the applied communication technology. In some examples, the implantable instrument 302 can be used to transmit communication signals to other devices, such as a charger or the handheld programmer, and the communication system 322 can be implemented to generate communication signals and provide the communication signals to the tether 306 for transmission via the interface system 304. In some examples, the communication system 322 can be configured to receive and transmit radiofrequency signals. The controller 324 can include a microcontroller to operate the recharge system 320 and to receive and operate in response to communication signals or generate communication signals from communication system 322. For example, the controller can cause the operation of the components of the fluidics circuit 316.

The components of the fluidics circuit 316 provide for the transfer of the fluid between the reservoir 308 and the inflatable member 310. The fluidics circuit 316 can include a pump assembly 330, a valve assembly 332, and a pressure sensor 334. The pump assembly 330 can include channels and tubing to direct fluid from one port to another port of the fluidics circuit. For example, the pump assembly 330 can include a manifold, which can be a hermetic manifold, to segment and contain the fluid from the electronics components and direct the fluid from one port in the fluid circuit 316 to another port and to reduce the chance of fluid or gas exchange. The pump assembly 330 can further include a pump, such as a plurality of pumps, to provide for the flow of fluid. In one example, the pump assembly 330 can include a pair of pumps that can operate independently of each other and typically do not operate simultaneously to provide for the inflation, and sometimes deflation, of the inflatable member 310. The pump assembly 330 is fluidically coupled to a valve assembly 332 that provided for selective sealing of a corresponding fluid channel and can maintain an inflated or deflated state. The pressure sensor 334 can be implemented to detect pressure within the fluid chamber or system and flow.

The interface system 304 can include a communication conductor 340, a power conductor 342, or both the communication conductor 340 and the power conductor 342. In one example, the communication conductor 340 is configured as antenna for radiofrequency communication. The communication conductor 340 can include a plurality of antennae for radiofrequency communication. For example, the communication conductor can include an antenna to receive radiofrequency signals in the 2.400-2.4835 GHz range for Bluetooth Low Energy communication. Additionally, or alternatively, the communication conductor 340 can include a coil or other structure for inductive communication. The power conductor 342 can include a coil or other conductor to receive inductive power transfer from a charger. In one example, the depth of the medical instrument implanted into the body can adversely affect charging more than communication, and the interface system 304 includes the power conductor 342 whereas the communication conductor is disposed with the medical instrument 302.

The elongated tether 306 is configured to mechanically couple to the interface system 304 at a distal end and to the medical instrument 302 at the proximal end. The elongated tether also includes lead conductor, such as a communication lead conductor to electrically couple the communication conductor 340 to the communication system 322, and a power lead conductor to electrically couple the power conductor 342 to the recharge system 320. The lead conductors can be encased in a biocompatible sheath, which may include a kink resistant surface. In still another example, the medical device 300 can include a plurality of interface systems, such as first interface system coupled via a first tether to the medical instrument and a second interface system coupled via a second tether to the medical instrument. In this example, the first interface system can include a power conductor and the second interface system can include a communication conductor.

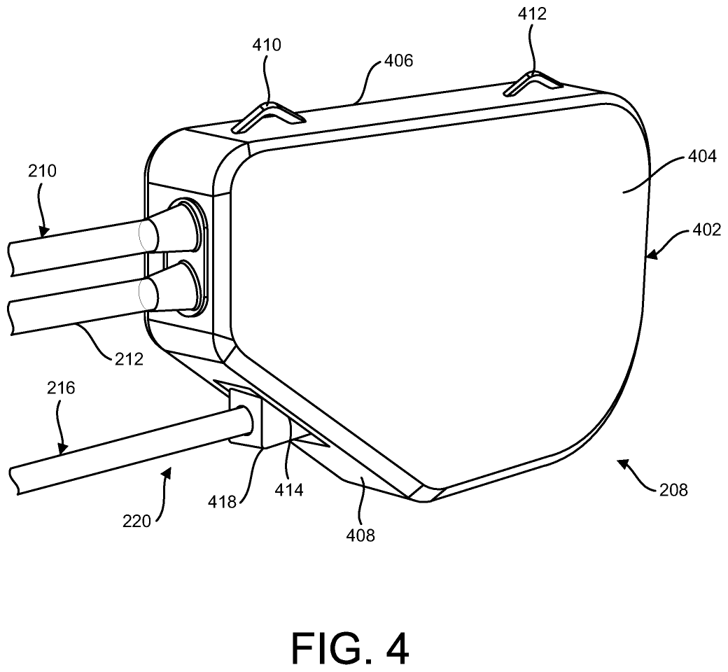

FIG. 4 illustrates a portion of the implantable medical device 200 featuring the electromechanical actuation device 208. The actuation device 208 includes a hermetically sealed housing 402 having a first side wall 404, a second side wall 406 opposite the first side wall 404, and a peripheral wall 408 extending between the first and second side walls 404, 406. The housing 402 defines an internal compartment defined by the space within the actuation device 208 that is bordered by the first side wall 404, the second side wall 406 and the peripheral wall 408. In some instances, the housing 402, and thus the first side wall 404, the second side wall 406 and the peripheral wall 408, are composed of a metallic material, such as, but not limited to, titanium. The internal compartment of the actuation device 208 is configured to carry rechargeable energy storage system 312, electronics components 314, and a fluidic circuit 316. In one example, the internal compartment may include a plurality of partitions that can be separated by walls and hermetically sealed from one another within the internal compartment. For example, the rechargeable energy storage system 312 and electronics components 314 may be carried in a first partition, and the fluidic circuit 316 may be carried in a second partition. The first partition may be hermetically sealed from the second partition. Feedthrough pins can be applied across the hermetic seal to allow the electronics components 314 to control the fluidic circuit 316. In one example, the electronics components 314 can be implemented by various components including resistors, capacitors, transistors, and integrated circuits disposed on one or more printed circuit boards within the internal compartment. The fluidic circuit 316 can be implemented via titanium manifolds and electromagnetic pumps or piezoelectric pumps. In one example, the housing 402 can including an attachment device, such as loops 410, 412 formed in peripheral wall 408 to receive sutures that can be applied to anchor the actuation device 208 to a structure within the patient.

The proximal end 220 of the tether 216 is mechanically coupled to the housing 402. In one example, an end region of the proximal end 220 of the tether 216 is disposed within the housing 402 through housing input 414 and held against the peripheral wall 408 via a strain relief 418. The end region of the proximal end 220 can be attached to the housing 402 via set screws or another attachment mechanism. The proximal end 220 can include an electrical contact for a power lead and an electrical contact for a communication lead, the electrical contacts can be electrically coupled to the electronic components via pins, flex circuits, or other electrical leads. In one example, housing input 414 is an opening to a third partition of the internal compartment, and the proximal end 220 is disposed within a third partition that is hermetically sealed from the first partition that includes the electronics components 314.

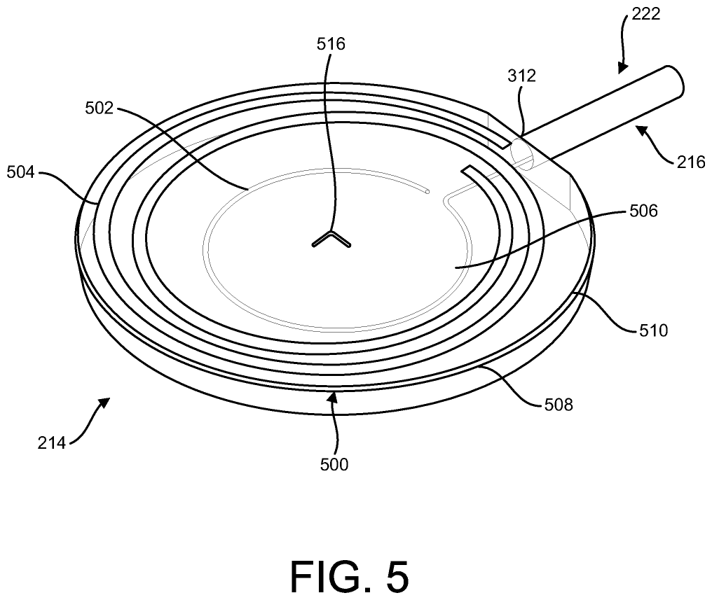

FIG. 5 illustrates a portion of the implantable medical device 200 featuring the power and communication interface system 214. The power and communication interface system 214 includes a puck 500 carrying a conductive communication antenna 502 and a conductive secondary coil 504. The communication antenna 502 can be configured to receive a transcutaneous communication signal, such as from a handheld programmer, via a radio frequency signal. The secondary coil 504 can be configured to receive an inductive power signal, such as from a charger. The puck 500 may include an additional antenna to receive additional radio frequency signals or an additional inductive coil, such as a coil coplanar with and disposed within the secondary coil 504, to receive inductive communication such as via inductive telemetry. The interface system 214 can include a first major surface 506, a second major surface 508 and a rim 510. In one example, the first and second major surfaces 506, 508 and rim 510 are formed in the puck 500. In the illustrated example, the communication antenna 502 and a secondary coil 504 are surrounded by and disposed within the puck 500 so that the coils are coplanar with a major surface 506, 508. In one example, the distal end 222 of the tether 216 is disposed within the puck 500 and includes a strain relief 512 at the rim 510. The puck 500 can be molded around the distal end 222 of the tether 216 to attach the tether 216 to the interface system 214, or the interface system 214 can be mechanically coupled to the tether 216 via another attachment mechanism. The distal end 222 can include an electrical contact for the power lead and an electrical contact for a communication lead. The communication antenna 502 can be conductively attached to the communication lead, and the secondary coil 504 can be conductively attached to the power lead.

The interface system can be hermetically sealed in one example. For example, the puck 500 can be formed of a ceramic material that can be generally transparent to signals. In examples in which the communication antenna 502 and secondary coil 504 are hermetically encased in the interface system 214, the communication antenna 502 and secondary coil 504 can be formed of copper and include circuit board materials. The use of copper in the antenna 502 and secondary coil 504 can reduce attenuation. When disposed in the puck 500, the copper antenna 502 and secondary coil 504 reduce attenuation due to implant depth. In one example, the hermetically sealed puck 500 can include circuit board elements as well, such as a Bluetooth communication module, that are operably coupled to electronic components in the actuation device 208 via the tether 216.

In examples in which the communication antenna 502 and the secondary coil 504 are not hermetically encased in the interface system 214, the communication antenna 502 and the secondary coil 504 are formed of a biocompatible material, such as a stamped titanium core to provide structure that is clad with gold or silver to provide conductivity. The puck 500 may be formed of a biocompatible and dielectric or insulative material, such as a polyurethane or silicone. The communication antenna 502 and secondary coil 504 can be encased in the biocompatible and dielectric or insulative material of the puck 500. In the illustrated example, the puck 500 is a thin disk formed to fit the size of the communication antenna 502 and secondary coil 504, but other configurations are contemplated. In the example, the puck 500 includes a diameter much larger than its thickness. The interface system 214, including the puck 500, communication antenna 502 and secondary coil 504 may be pliable or flexible rather than rigid, or the interface system 214 may include pliable and flexible portions to permit bending or flexing when the patient moves so as not to cause discomfort. In one example, the interface system 214 can including an attachment device, such as loops formed in puck 500 to receive sutures that can be applied to anchor the interface system 214 to a structure within the patient. In another example, the interface system 214 can include a mesh layer 514 attached to the puck at a major surface 508 to provide a coupling with tissue and hold the interface system 214 in place with respect to the patient. Additionally, the interface 214 may be textured and include a formation 516, such as a protrusion in the puck 500 at a major surface 506, to allow detection or location of the interface system 214 for coupling with the charger. In some examples, the interface system 214 may include a layer of flexible, such as scored, ferrite in a plane generally parallel to the secondary coil 504 to enhance inductive power transfer.

In one example, the actuation device 208, tether 216, and interface system 214 are integrated together as a single apparatus. For example, the distal end 222 of the tether 216 is integrally formed with the interface system 214, and the proximal end 220 of the tether 216 is integrally formed with the housing 402. In another example, the tether 216 may be integrally formed with the interface system 214, and the size of the interface and length of the tether may be selected by a clinician and attached, such as removably attached, to the actuation device during surgery. For example, the surgeon may attach the proximal end 220 of the tether 216 to the actuation device via the housing input 414, which may be held in place with a set screw or other attachment mechanism, and the strain relief 418 can be attached to the housing 402. The integrated interface system 214 and tether 216 may be offered in different sizes of interface and lengths of tether, and the clinician can select the appropriate size and length to suit the patient or the application.

In certain applications, the actuation device 208 may be deeply imbedded in a patient such as in the retropubic space and may not be conducive to inductive charging or in an area which may be comfortable to wear a charger during charging. The interface 214 may extend from the actuation device 208 and be placed closer to the skin of the patient or in an area which may be better suited for charging or communication than the actuator device 208 such as near the skin in the abdomen or the back. The patient can receive effective therapy with the actuation device 208 without having to compromise on communication or charging capabilities, or receive effective communication and charging capabilities without having to compromise therapy when the location of the actuation device makes charging and communication relatively difficult.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. An implantable medical device, comprising:
a housing forming an internal compartment;
electronic components disposed within the internal compartment;
an interface system configured to receive at least one of power signals and wireless electrical communication signals; and
an elongated tether having a conductive lead, wherein the tether mechanically couples the housing to the interface system and allows the housing and interface system to be implanted at spaced-apart locations within a patient, further wherein the conductive lead electrically couples the interface system to the electronic components.

2. The implantable medical device of claim 1, wherein the electronic components include a treatment system, a communication system, and a recharge system, and the recharge system is coupled to a rechargeable power source.

3. The implantable medical device of claim 2, wherein the rechargeable power source is a battery disposed within the internal compartment.

4. The implantable medical device of claim 1, and further comprising a fluidic circuit disposed within the internal compartment.

5. The implantable medical device of claim 4, wherein the fluidic circuit includes a pump assembly in fluid communication with a valve assembly and a pressure sensor.

6. The implantable medical device of claim 4, wherein the internal compartment includes a plurality of partitions including a first partition and a second partition, the fluidic circuit disposed within the first partition and the electronics components disposed in the second partition, wherein the first partition is hermetically sealed from the second partition.

7. The implantable medical device of claim 6, wherein the tether includes a proximal end and a distal end, wherein the distal end is mechanically coupled to the interface system and the proximal end is received within a partition of the plurality of partitions and mechanically coupled to the housing.

8. The implantable medical device of claim of claim 7, wherein the conductive lead is electrically coupled to electronic components via feedthrough pins coupled to the proximal end.

9. The implantable medical device of claim 1, wherein the interface system includes a coil configured to receive the power signals and an antenna configured to receive the wireless electrical communication signals.

10. The implantable medical device of claim 9, wherein the coil and the antenna are disposed within a ceramic puck.

11. The implantable medical device of claim 10, wherein the puck includes a first major surface and an opposite second major surface, and wherein the first major surface is coupled to a mesh layer.

12. The implantable medical device of claim 11, wherein the second major surface is textured to form a protrusion.

13. The implantable medical device of claim 10, wherein coil and antenna are disposed within a flexible puck formed of a polyurethane or silicone.

14. The implantable medical device of claim 1, wherein the housing is configured to be implanted in a first location of a patient and the interface system is configured to be implanted in a second location of the patient.

15. A method to implant an implantable medical device within a patient, the method comprising:
providing an implantable medical device, the implantable medical device including:
a housing forming an internal compartment;
electronic components and a rechargeable power source disposed within the internal compartment, the electronic components including a treatment system, a communication system and a recharge system, the recharge system coupled to the rechargeable power source;
an interface system including at least one of a coil configured to receive power signals and an antenna configured to receive wireless electrical communication signals; and
an elongated tether having a conductive lead, wherein the tether mechanically couples the housing to the interface system and allows the housing and interface system to be implanted at spaced-apart locations within a patient, further wherein the conductive lead electrically couples the at least one of the coil and the antenna to the electronic components;
implanting the housing in a first location within the patient; and
implanting the interface system at a second location with the patient and spaced-apart from the first location.

16. The method of claim 15, wherein the tether is integrally formed with the interface system, and further comprising coupling the tether to the electronic components.

17. An implantable urological device, comprising:
a reservoir configured to receive a fluid;
an inflatable member in fluid communication with the reservoir;
an implantable medical instrument in fluid communication with the reservoir and the inflatable member, the implantable medical instrument comprising:
a housing forming an internal compartment;
a fluidic circuit disposed within the internal compartment, the fluidic circuit including a pump assembly configured to transfer the fluid from the reservoir to the inflatable member; and
electronic components operably coupled to the fluidic circuit and configured to operate the fluidic circuit, the electronic components coupled to a rechargeable power source, the electronics components disposed within the internal compartment;
wherein the electronic components include a communication system and a recharge system disposed within the internal compartment, the recharge system coupled to the rechargeable power source;
an interface system including at least one of a coil and an antenna, the coil configured to receive power signals, and the antenna configured to receive electrical communication signals; and
an elongated tether having a conductive lead, wherein the tether mechanically couples the housing to the interface system and allows the housing and interface system to be implanted at spaced-apart locations within the patient, further wherein the conductive lead electrically couples the at least one of the coil and the antenna to the electronic components.

18. The implantable urological device of claim 17, wherein the inflatable member includes a plurality of inflatable cylinders in fluid communication with the medical instrument and the reservoir, the plurality of inflatable cylinders configured to be disposed within a corpora cavernosa of a penis.

19. The implantable urological device of claim 17, wherein the inflatable member includes an inflatable cuff configured to be disposed about a urethra.

20. The implantable urological device of claim 17, wherein the interface system is integrally formed with the tether, and the tether is removably coupleable to the medical instrument.
